(19) United States
(12) Patent Application Publication
(10) Pub. No.: US 2010/0210932 A1
(43) Pub. Date: Aug. 19, 2010

(54) METHOD OF ANALYZING AN ANALYTE

(75) Inventor: Mihailo V. Rebec, Bristol, IN (US)

Correspondence Address: NIXON PEABODY LLP, 300 S. Riverside Plaza, 16th Floor, CHICAGO, IL, 60606-6613, US

(73) Assignee: Bayer HealthCare LLC, Tarrytown, NY (US)

(21) Appl. No.: 12/531,634

(22) PCT Filed: Dec. 21, 2007
(86) PCT No.: PCT/US07/26236

§ 371 Date: Apr. 26, 2010

Related U.S. Application Data
(60) Provisional application No. 60/919,116, filed on Mar. 20, 2007.

(51) Int. Cl. A61B 5/145

(52) U.S. Cl. 600/347

(57) ABSTRACT

A method of using a diffusion-based, continuous-monitoring system to analyze an analyte includes creating at least one diffusion channel in an area of skin. The diffusion channel is maintained for a desired duration. The levels of the analyte are continuously monitored for the desired duration via a diffusion-based, continuous-monitoring device. The levels of the analyte at the area of skin are analyzed to determine if a condition associated with the analyte is present.

### FIELD OF THE INVENTION

The present invention relates generally to a method of analyzing for an analyte and, more specifically, to a method of diffusion-based, continuous analyte analyzation.

### BACKGROUND OF THE INVENTION

The quantitative determination of analytes in body fluids is of great importance in the diagnoses and maintenance of certain physiological abnormalities. For example, lactate, cholesterol and bilirubin should be monitored in certain individuals. Additionally, determining glucose in body fluids is important to diabetic individuals who must frequently check the glucose level in their body fluids to regulate the glucose intake in their diets. The results of such tests can be used to determine how much, if any, insulin or other medication needs to be administered. Analytes may be continuously monitored to obtain a number of readings over a desired period of time.

It would be desirable to have a method of continuously monitoring an analyte that would be performed in an efficient manner.

### SUMMARY OF THE INVENTION

According to one method, a diffusion-based, continuous-monitoring system to analyze an analyte includes creating at least one diffusion channel in an area of skin. The at least one diffusion channel is maintained for a desired duration. The levels of the analyte are continuously monitored for the desired duration via a diffusion-based, continuous-monitoring device. The levels of the analyte at the area of skin are analyzed to determine if a condition associated with the analyte is present.

According to another method, a diffusion-based, continuous-monitoring system to analyze an analyte includes providing a diffusion-based, continuous-monitoring device. The device includes a communications interface that is adapted to connect with a receiving module via a communications link. At least one diffusion channel is created in an area of skin. The at least one diffusion channel is maintained for a desired duration. The levels of the analyte are continuously monitored for the desired duration via the diffusion-based, continuous-monitoring device. The levels of the analyte at the area of skin are analyzed to determine if a condition associated with the analyte is present.

### BRIEF DESCRIPTION OF THE DRAWINGS

FIG. 1 is a diffusion-based, continuous-monitoring system shown in a transdermal application according to one embodiment.

FIG. 2 is the continuous-monitoring system of FIG. 1 being connected to a receiving module.

### DETAILED DESCRIPTION OF THE ILLUSTRATED EMBODIMENTS

The present invention is directed to a method of using a diffusion-based, continuous-monitoring system to analyze for at least one analyte in an area of the skin. By continuously monitoring the level of an analyte at an area of skin, it can be determined whether action needs to be taken by the individual to the condition.

Analytes that may be measured using the present invention include glucose, lipid profiles (e.g., cholesterol, triglycerides, LDL and HDL), microalbumin, hemoglobin A_{IC}, fructose, lactate, or bilirubin. The present invention is not limited, however, to these specific analytes and it is contemplated that other analyte concentrations may be determined. The analytes may be in, for example, a whole blood sample, a blood serum sample, a blood plasma sample, or other body fluids like ISF (interstitial fluid) and urine.

The term “level” is defined herein as including any information related to, for example, the amount, relative concentration and absolute concentration. The term “level” as defined herein also includes changes in the amount, relative and absolute concentrations, whether in a percentage or absolute context. These “level” changes may be used over a selected duration of time such as, for example, a time change in amount or concentration. The “level” may refer to a time change in amount or concentration, and compared to a later time change. The amount and rate of change of these analytes are powerful tools in assessing the physiological state of the individual.

According to one method, at least three criteria may be considered in selecting a suitable diffusion-based, continuous-monitoring system to analyze analytes in a body fluid sample from an area of skin. First, a diffusion-enhancing process for the skin is selected. Second, a material is selected to assist in maintaining contact with the skin and further enhance diffusion of the analyte in the body fluid sample from an area of skin. Third, a diffusion-based, continuous-monitoring system is selected to monitor the analyte in the body fluid sample that are diffused from the skin.

According to one method, the diffusion-enhancing process for the skin is selected based on factors such as the following: length of time of testing, the analyte (e.g., glucose) to be analyzed, and the area of the skin from where the analytes are located. It is desirable for the diffusion-enhancing process to maintain the diffusion channel throughout the desired time period.

Skin abrasion is typically selected when the continuous-testing period is a relatively short period of time (e.g., less than about 8 hours). Skin abrasion is desirable for a shorter continuous-testing period because of the minimum impact on the skin. It is contemplated that a number of skin-abrasion techniques may be used. In one technique, skin abrasion occurs using a gel material including pumas or other skin-abrasion materials. In this technique, the gel material including pumas or other skin-abrasion materials is rubbed on the skin to increase the permeability of the skin. Skin abrasion may occur by other techniques such as using a generally coarse material (e.g., sandpaper), tape peeling or pumas paper.

To increase the porosity of skin (e.g., the stratum cornium, epidermis and/or dermis), chemical agents and physical agents may be used. The chemical and physical agents desirably assist in breaking down the lipids on the stratum cornium. The chemical and physical agents are typically used in short-term solutions and medium-term solutions. It is contemplated, however, that the chemical and physical agents may be used in long-term solutions.

The chemical agents may be skin hydration or skin exfoliates that increase the hydration and porosity of the skin. Skin hydration/exfoliates may include those commercially used in skin products. Some non-limiting examples of chemical agents that may be used include d-limonene, L-limonene, and alpha-terpinene. These chemical agents act by extracting lipids from, for example, the stratum cornium, which result in the disruption of the stratum cornium and desquamated stratum cornium flake.

There are number of physical processes that can be used to enhance the permeability of the skin so as to increase the diffusion of the analytes of interest. In one process, needle-less jet injectors are used with very fine, particulates of inert material that are fired directly into the skin using high-pressure gas. In another process, pulsed magnetic fields may be used to create transient pores in the skin, resulting in increased permeation. It is contemplated that other physical processes may be used to enhance the permeability of the skin.

If the continuous-testing period is longer (e.g., from about 8 hours to 24 hours), then a different diffusion-enhancing approach may be selected. For such a period, various approaches may be selected such as microporation, microneedle-diffusion enhancement, pressure members, multiple lances, heavier abrasions and ultrasound energy.

In one method, a microporation or a microneedle-diffusion enhancement approach may be used for longer continuous testing periods. A microporation approach creates sub-millimeter size apertures in the epidermis. In one microporation technique, a laser-poration technique may be used to deliver laser power directly to the skin to create apertures or pores. Laser-poration techniques are typically used to form shallow apertures or pores.

In a further method, a series of absorbing dots is located in the stratum cornium and then followed by delivery of a laser that absorbs and softens at each point. The absorbent material converts the laser power to heat, which combined with pressure, create the apertures in the stratum cornium.

A microneedle-diffusion enhancement approach creates apertures in the epidermis and dermis. In another method, a pressure member is adapted to apply pressure to and stretch the skin in preparation for forming a tear in the skin. In another approach, a heavier abrasion of the skin could be performed such as using a more coarse material. An example of a more coarse material includes, but is not limited to, coarser sandpaper.

In another method, ultrasound energy is used to disrupt the lipid bilayer of the stratum cornium so as to increase the skin permeability. Ultrasound energy typically forms shallow apertures. By increasing the skin permeability, the amount of interstitial fluid (ISF) used in monitoring the analytes is increased. One non-limiting source of an ultrasound energy system is Sontra SonoPrep® ultrasonic skin permeation system marketed by Sontra Medical Corporation. The SonoPrep® system applies relatively low frequency ultrasonic energy to the skin for a limited duration (from about 10 to 20 seconds). The ultrasonic horn contained in the device vibrates at about 55,000 times per second (55 KHz) and applies energy to the skin through the liquid medium (e.g., hydrogel or liquid) to create cavitation bubbles that expand and contract in the liquid medium.

The chemical and physical agents discussed above in the generally short term can also be used in medium continuous-testing periods to increase and maintain the porosity of the skin. It is contemplated, however, that the chemical and physical agents may be used to obtain longer term action. For example, delipidating agents may be used in combination with physical agents such as ultrasonic preparation to create more long term diffusional channels.

If the continuous-testing period is even longer (e.g., at least 24 hours to about 48 hours), a deep, laser-ablation technique or lance may be selected. A deep, laser-ablation technique is desirable because the monitoring process can function longer due to the time needed to close the aperture created in the skin. The laser-ablation technique typically forms wide apertures. It is contemplated that a microneedle diffusion-enhancing approach, laser poration or lancets may also be used to provide a deeper aperture.

The size of the analyte to be analyzed may also affect the diffusion-enhancing technique to be used. If the analyte is a larger molecule, the diffusion-enhancing process would desirably form a larger aperture in the skin. Similarly, if smaller analytes are to be monitored, the diffusion-enhancing process desirably would form a smaller aperture in the skin.

The area of the skin where the analyte is located is also a consideration in selecting the diffusion-enhancing process. For example, if the epidermis or the upper part of the dermis is where the analyte is to be monitored, the diffusion-enhancing process would be selected to disrupt the stratum cornium. Examples of such diffusion-enhancing processes include skin abrasion, skin hydrations (which increase the hydration of the skin) and skin exfoliates.

If monitoring of the analyte in the ISF of the lower dermis is desired, the diffusion-enhancing process is selected to create diffusion channels deep into the dermis. If monitoring of the analyte in the ISF or the subcutaneous region is desired, the diffusion-enhancing process is selected to create diffusion channels through the dermis into the subcutaneous region. Non-limiting examples of diffusion-enhancing processes that create deep diffusion channels into the dermis or subcutaneous region include, but are not limited to, laser poration, microneedles and lancets. It is also contemplated that an electric discharge with high energy and conductivity may also be used to create deep diffusion channels.

The chemical and physical agents discussed above in the generally short term may also be used in longer continuous-testing periods to increase and maintain the porosity of the skin.

In addition to selecting a continuous diffusion-enhancing method, a material is selected to assist in maintaining contact with the skin and to match the monitoring requirements in one method. The diffusion-enhancing material maintains desirable skin contact at all times and assists in maintaining the diffusion channel. The material may be selected based on factors such as the following: length of monitoring time, the analyte to be monitored, and the area of the skin from where the analytes are located. For example, the viscosity of the material may be matched with the analytes to be monitored. More specifically, the viscosity would be the choice of material based on the size of the desired analyte. For example, if changes in the potassium level are being monitored, a small porosity, high viscosity material is typically desirable since the diffusion rates of potassium are relatively fast. In another example, if changes in a relatively large analyte are being monitored, then a low viscosity material would be typically selected.

Examples of diffusion-enhancing materials that may be used in the diffusion-based, continuous-monitoring system include, but are not limited to, hydrogels, liquids and a liquid-stabilizing layer containing a liquid or hydrogel. The diffusion-enhancing material also desirably assists in hydrating the skin and maintaining an opening in the skin. By maintaining the opening, a liquid bridge is formed such that the analyte diffuses from a layer in the skin through the opening. The liquid bridge may be between a hydrogel/liquid and a body fluid such as ISF (interstitial fluid) or a whole blood sample.

The hydrogels typically have high water content and tacky characteristics. Hydrogels assist in carrying the analyte to the skin surface and hydrating the skin. Hydrogels are typically used with smaller sized analytes, shorter analysis times and an upper dermis analysis site.

A hydrogel composition is defined herein as including a cross-linked polymer gel. The hydrogel composition generally comprises at least one monomer and a solvent. The solvent is typically substantially biocompatible with the skin. Non-limiting examples of solvents that may be used in the hydrogel composition include water and a water mixture. The amount of solvent in the hydrogel is generally from about 10 to about 95 weight percent and may vary depending on the monomer amount, crosslinking, and/or the desired composition of the gel. One non-limiting example of a hydrogel/liquid is dimethylsulfoxide (DMSO). DMSO also assists in solubilizing lipids. Examples of a liquid that may be used include alcohol in combination with water. It is contemplated that other hydrogels/liquids may be used.

The hydrogel/liquid may be located in a material (i.e., a liquid-stabilizing layer). This material may be selected to assist in maintaining contact with the skin as well as being able to retain the hydrogel/liquid. The liquid-stabilizing layer may include a chamber where the analytes of interest can diffuse. One non-limiting example of a material that can be used is a sponge or spongy material. The spongy material includes unbound liquid such as water and provides some structure to the unbound water. The spongy material is typically used with larger sized analytes, longer monitoring time and deeper monitoring sites.

The amount of hydrogel that is selected is based on the need to provide a hydrated skin and having the hydrogel remain in intimate contact with the skin. One disadvantage of using a large amount of hydrogel is the potential impact on the lag time of the analyte diffusing to the diffusion-based, continuous-monitoring system and/or the analysis components reaching the skin. Such occurrences may potentially impact the analysis time.

Other materials may be used to create content with skin and conduct further analysis. Materials include, but are not limited to, woven materials, non-woven materials, and polymeric films with apertures or porations formed therein. The polymeric films may, for example, be cast polymeric films. These materials may be used with liquids to facilitate diffusion of the analytes from the skin.

Additives may be added to the hydrogel or liquid. For example, to assist in dissolving lipids, the hydrogel or liquid may include SDS (sodium dodecyl (lauryl) sulfate) or SLS (sodium lauryl (laureth) sulfate). It is contemplated that other additives may be included in the hydrogel or liquid to assist in dissolving the lipids such as soaps. In another embodiment, DMSO may be used as an additive to another hydrogel/liquid to assist in solubilizing lipids. Additional analysis components may also be added to the hydrogels/liquids.

In another embodiment, an interference-filtering component may be added to the hydrogels/liquids. These interference-filtering components may include size exclusion, interference-binding molecules, and/or molecules that remove or convert interfering substances. Some non-limiting examples of interference-binding molecules are antibodies or materials with appropriate charges. Another example is changing the ionic charge nature of the hydrogel or diffusion matrix such that charged interference molecules are inhibited from getting to the surface of the sensor.

Hypertonic solutions, hypotonic solutions and buffered solutions may be used as a diffusion-enhancing material. Hypertonic solutions are solutions having a high solute concentration, while hypotonic solutions are solutions having a low solute concentration. Hypertonic solutions assist in driving up the body fluid (e.g., ISF) closer to the skin surface. Hypotonic solutions, on the other hand, assist in driving up the analytes closer to the skin surface. The hypertonic or hypotonic solutions in one embodiment may be included with the hydrogel or liquid.

To assist in analyzing the analytes of interest, a charged additive may be added to the hydrogel or liquid. In one embodiment, a cationic surfactant is added to the hydrogel or liquid. In another example, an anionic surfactant is added to the hydrogel or liquid.

It is contemplated that other additives may be added to the hydrogel or the liquids to assist in monitoring the analytes.

A diffusion-based, continuous-monitoring device is selected that monitors the analyte of the body fluid sample that is diffused from the skin. The diffusion-based, continuous-monitoring device may be selected from an electrochemical-monitoring system, an optical-monitoring system, an osmotic-monitoring system and a pressure-based monitoring system. A pressure-based monitoring system includes systems associated with the binding of an analyte by components of the hydrogel, which results in a volume change in the gel. The monitoring may be performed in a vertical or horizontal direction with respect to the diffusion channel(s) formed in the skin. It is contemplated that the analyte may be carried out in the material that is selected to assist in maintaining contact with the skin (e.g., the hydrogel or liquid).

The diffusion-based, continuous-monitoring device is typically located near or at the skin. The diffusion-based, continuous-monitoring device may be coupled with the skin and is typically in intimate contact with the skin. For example, the diffusion-based, continuous-monitoring device may be adhered to the skin with an adhesive. The adhesive may be the hydrogel itself. In another embodiment, the adhesive is a separate component whose sole function is to adhere the continuous-monitoring device to the skin. In a further method, the diffusion-based, continuous-monitoring device may be coupled to the skin by a mechanical attachment. For example, the mechanical attachment may be a wrist band (e.g., an elastic band, a watch band, a band with an attachment mechanism such as a hook and loop mechanism). One example of a hook and loop mechanism is a Velcro® strap marketed by 3M Corporation of St. Paul, Minn. It is contemplated that other mechanical attachments may be used to couple or attach the continuous-monitoring device with skin.

The diffusion-based, continuous-monitoring device may have a variety of forms. For example, the continuous-monitoring device may be a pad, circular disk, polygonal shaped or non-polygonal shaped. The continuous-monitoring system may include the analysis element. For example, a pad with an analysis element may be used instead of, or in addition to, the analysis element being initially located in the hydrogel or liquid. In one embodiment, the analysis component may be initially located in the continuous-monitoring device.

In one embodiment, the diffusion-based, continuous-monitoring device includes a processor to process the data, a memory that stores data, and a communications interface. The data may be stored at regular intervals such as, for example, every minute, every 5 minutes or every 30 minutes. The intervals may be shorter such as every second or longer such as being several hours apart. The selected intervals depend on the analyte being tracked and the rate of change of that analyte. It is contemplated that other regular or non-regular intervals may be used to store the data.

The data may be any information that assists in monitoring the analytes. This typically includes the level of analytes, but may include other information. This information may then be processed to determine a course of action to address the condition. By storing the data in the continuous-monitoring device, this data can be accessed and used to assist in monitoring the analyte. It is desirable for the continuous-monitoring device to tabulate, transmit and store information that assists in monitoring the analyte.

In one embodiment, the continuous-monitoring device is connected to a remote-monitoring system over a communications link. The communications link between the continuous-monitoring device and the remote-monitoring system may be wireless, hard wired or a combination thereof. The wireless communications link may include an RF link, an infrared link or an inductive magnetic link. The wireless implementation may include an interne connection. The continuous-monitoring device may communicate via its communication interface with devices such as a computer, e-mail server, cell phone or telephone. It is contemplated that the continuous-monitoring device may include other devices that are capable of storing, sending and/or receiving information.

The remote-monitoring system enables an individual such as a physician to monitor, for example, the level of the analyte from a remote location. The remote-monitoring system may be located in, for example, a hospital. The physician may be able to access information from the continuous-monitoring device via its communications interface using, for example, a computer or telephone. The remote-monitoring system is especially desirable for patients who are less lucid and need assistance with monitoring selected analytes. It is desirable for the remote-monitoring system to be able to display, calibrate and store information received from the continuous-monitoring device.

In one method, the continuous-monitoring device may forward information over a communications link in real-time. In another method, the continuous-monitoring device may store and process the data before forwarding the information over a communications link in another embodiment.

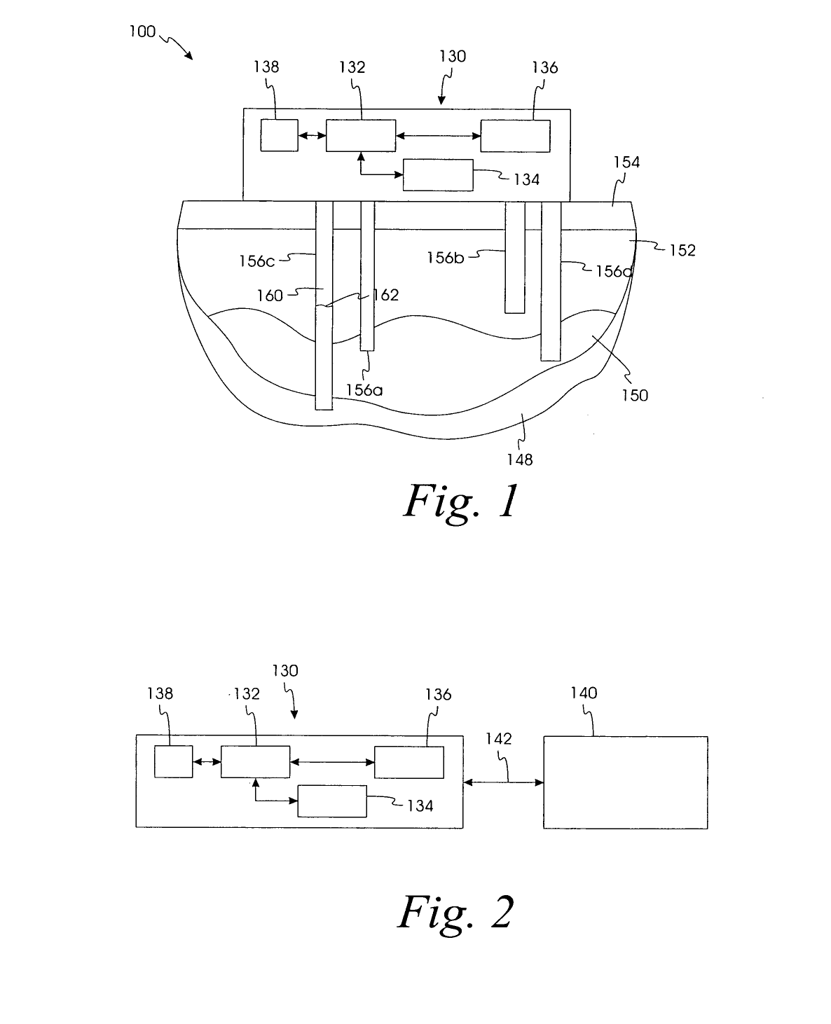

Referring to FIG. 1, a diffusion-based, continuous-monitoring system 100 is shown in a transdermal application. The continuous-monitoring system 100 includes a continuous-monitoring device 130 being placed above skin. The continuous-monitoring device 130 of FIG. 1 includes a processor 132, memory 134, a communication interface 136 and an analysis component 138. Referring to FIG. 2, the continuous-monitoring device 130 is shown in communication with a receiving module 140 (e.g., a remote-monitoring station) over a communications link 142.

The skin as shown in FIG. 1 includes a subcutaneous layer 148, a dermis layer 150, an epidermis layer 152 and a stratum cornium layer 154. The stratum cornium layer 154 has a plurality of channels 156a-d formed therein. The plurality of channels 156a-d may be formed by different methods such as discussed above. The channels may be of different sizes and depths depending on the analytes being analyzed and the location of the analytes in the skin. The analytes of interest may be located in the different layers of the skin. The analytes of interest are primarily located in the dermis layer 150, epidermis layer 152, or the subcutaneous layer 148. For example, analytes such as glucose, electrolytes and cholesterol are generally found in the epidermis. The hydrogel/liquid assists in diffusing the analytes to the surface of the skin. The channel 156c is shown with hydrogel/liquid 160.

In one method, a hydrogel/liquid is used to assist in diffusing the analyte to the surface of the skin. The channel 156c is shown with hydrogel/liquid 160. An interface 162 is formed between the hydrogel/liquid and the body fluid. The analysis may be performed in several locations in the continuous-monitoring system 100. For example, the analysis may be performed using the analysis components 138 in the continuous-monitoring device 130. The analysis components may include components such as a sensor, an enzyme or reagent, potentiostat, electrochemical analysis components (e.g., plurality of electrodes, etc.) and/or optical analysis components (e.g., light source, detector, etc.). In another example, the analysis may be performed on the skin and/or in the channels. It is contemplated that the analysis may take place in more than one location. For example, the hydrogel/liquid may include an analysis portion (e.g., a reagent or enzyme) that reacts with analyte in the channel, while the remainder of the analysis takes place on the skin or in the continuous-monitoring device 130.

According to one process, a technician programs the diffusion-based, continuous-monitoring device for operation. The technician may program, for example, the analyte to be monitored and the length of time of the monitoring. The technician may then proceed to form apertures in the skin to form the desired diffusion channels as discussed above for the desired time period. The technician locates the continuous-monitoring device on the individual. In one method, the technician locates the continuous-monitoring device on the arm. It is contemplated that the technician may locate the continuous-monitoring device on other locations. The continuous-monitoring device is adapted to process, calibrate, display, store and/or transmit information related to the analytes.

### Process A

A method of using a diffusion-based, continuous-monitoring system to analyze an analyte, the method comprising the acts of:

creating at least one diffusion channel in an area of skin;

maintaining the at least one diffusion channel for a desired duration;

continuously monitoring the levels of the analyte for the desired duration via a diffusion-based, continuous-monitoring device; and

analyzing the levels of the analyte at the area of skin to determine if a condition associated with the analyte is present.

### Process B

The method of process A wherein the at least one diffusion channel is a plurality of diffusion channels.

### Process C

The method of process A wherein the at least one diffusion channel is created by skin abrasion, microporation, microneedle-diffusion enhancement, pressure members, a lancet, ultrasound energy or laser ablation.

### Process D

The method of process A wherein the continuous time period is at least 8 hours.

### Process E

The method of process A wherein the continuous time period is at least 24 hours.

### Process F

The method of process A wherein the diffusion-based, continuous-monitoring system is an electrochemical-monitoring system.

### Process G

The method of process A wherein the diffusion-based, continuous-monitoring system is an optical-monitoring system.

### Process H

The method of process A further including storing the levels of the analyte.

### Process I

The method of process A further including topographically applying a hydrogel or liquid on the skin to assist in enhancing the diffusion of the analyte and positioning the diffusion-based, continuous monitoring device in communication with the hydrogel or liquid.

### Process J

The method of process I wherein the hydrogel or liquid includes a diagnostic element to assist in analyzing the levels of the analyte at the area of skin.

### Process K

The method of process I wherein positioning the monitoring device includes attaching the monitoring device to the skin.

### Process L

The method of process A further including displaying the levels of the analyte on the continuous-monitoring device.

### Process M

The method of process A wherein the analyte is glucose.

### Process N

A method of using a diffusion-based, continuous-monitoring system to analyze an analyte, the method comprising the acts of:

providing a diffusion-based, continuous-monitoring device, the device including a communications interface that is adapted to connect with a receiving module via a communications link;

creating at least one diffusion channel in an area of skin;

maintaining the at least one diffusion channel for a desired duration;

continuously monitoring the levels of the analyte for the desired duration via the diffusion-based, continuous-monitoring device; and

analyzing the levels of the analyte at the area of skin to determine if a condition associated with the analyte is present.

### Process O

The method of process N further including transmitting information directed to the levels of the analyte to the receiving module via the communications link.

### Process P

The method of process O further including receiving instructions from the receiving module via the communications link.

### Process Q

The method of process O wherein the transmitting of information is performed on a wireless system.

### Process R

The method of process O wherein the transmitting of information is performed on a wired system.

### Process S

The method of process O wherein the transmitting of information occurs at intervals between 5 minutes and 4 hours.

### Process T

The method of process N wherein the at least one diffusion channel is a plurality of diffusion channels.

### Process U

The method of process N wherein the at least one diffusion channel is created by skin abrasion, microporation, microneedle-diffusion enhancement, pressure members, a lancet, ultrasound energy or laser ablation.

### Process V

The method of process N wherein the continuous time period is at least 8 hours.

### Process W

The method of process N wherein the continuous time period is at least 24 hours.

### Process X

The method of process N wherein the diffusion-based, continuous-monitoring system is an electrochemical-monitoring system.

### Process Y

The method of process N wherein the diffusion-based, continuous-monitoring system is an optical-monitoring system.

### Process Z

The method of process N further including storing the levels of the analyte.

### Process AA

The method of process N further including displaying the levels the analyte.

### Process AB

The method of process N wherein the analyte is glucose.

While the present invention has been described with reference to one or more particular embodiments, those skilled in the art will recognize that many changes may be made thereto without departing from the spirit and scope of the present invention. Each of these embodiments, and obvious variations thereof, is contemplated as falling within the spirit and scope of the invention as defined by the appended claims.

1. A method of using a diffusion-based, continuous-monitoring system to analyze an analyte, the method comprising the acts of:
creating at least one diffusion channel in an area of skin;
maintaining the at least one diffusion channel for a desired duration;
continuously monitoring the levels of the analyte for the desired duration via a diffusion-based, continuous-monitoring device; and
analyzing the levels of the analyte at the area of skin to determine if a condition associated with the analyte is present.

2. The method of claim 1, wherein the at least one diffusion channel is a plurality of diffusion channels.

3. The method of claim 1, wherein the at least one diffusion channel is created by skin abrasion, microporation, microneedle-diffusion enhancement, pressure members, a lancet, ultrasound energy or laser ablation.

4. The method of claim 1, wherein the continuous time period is at least 8 hours.

5. The method of claim 1, wherein the continuous time period is at least 24 hours.

6. The method of claim 1, wherein the diffusion-based, continuous-monitoring system is an electrochemical-monitoring system.

7. The method of claim 1, wherein the diffusion-based, continuous-monitoring system is an optical-monitoring system.

8. The method of claim 1, further including storing the levels of the analyte.

9. The method of claim 1, further including topographically applying a hydrogel or liquid on the skin to assist in enhancing the diffusion of the analyte and positioning the diffusion-based, continuous monitoring device in communication with the hydrogel or liquid.

10. The method of claim 9, wherein the hydrogel or liquid includes a diagnostic element to assist in analyzing the levels of the analyte at the area of skin.

11. The method of claim 9, wherein positioning the monitoring device includes attaching the monitoring device to the skin.

12. The method of claim 1, further including displaying the levels of the analyte on the continuous-monitoring device.

13. The method of claim 1, wherein the analyte is glucose.

14. A method of using a diffusion-based, continuous-monitoring system to analyze an analyte, the method comprising the acts of:
providing a diffusion-based, continuous-monitoring device, the device including a communications interface that is adapted to connect with a receiving module via a communications link;
creating at least one diffusion channel in an area of skin;
maintaining the at least one diffusion channel for a desired duration;
continuously monitoring the levels of the analyte for the desired duration via the diffusion-based, continuous-monitoring device; and
analyzing the levels of the analyte at the area of skin to determine if a condition associated with the analyte is present.

15. The method of claim 14, further including transmitting information directed to the levels of the analyte to the receiving module via the communications link.

16. The method of claim 15, further including receiving instructions from the receiving module via the communications link.

17. The method of claim 15, wherein the transmitting of information is performed on a wireless system.

18. The method of claim 15, wherein the transmitting of information is performed on a wired system.

19. The method of claim 15, wherein the transmitting of information occurs at intervals between 5 minutes and 4 hours.

20. The method of claim 14, wherein the at least one diffusion channel is a plurality of diffusion channels.

21-28. (canceled)
